(19) United States
(12) Patent Application Publication
(10) Pub. No.: US 2026/0083565 A1
(43) Pub. Date: Mar. 26, 2026

(54) DRY HYDROGEL IMPLANTS

(71) Applicant: Sparta Biomedical, Inc., Madison, NJ (US)

(72) Inventors: Benjamin J. WILEY, Durham, NC (US); Dushyanth SURAKANTI, Madison, NJ (US)

(21) Appl. No.: 19/113,420

(22) PCT Filed: Sep. 19, 2023
(86) PCT No.: PCT/US2023/074611

§ 371 Date: Mar. 19, 2025

Related U.S. Application Data
(60) Provisional application No. 63/408,087, filed on Sep. 19, 2022.

(51) Int. Cl. A61F 2/30; A61L 27/16; A61L 27/20; A61L 27/52

(52) U.S. Cl. CPC A61F 2/30756; A61F 2/3094; A61L 27/16; A61L 27/20; A61L 27/52; A61F 2002/30751; A61F 2002/3084; A61L 2430/02

(57) ABSTRACT

Dry cellulose-reinforced hydrogels may include a cellulose nanofiber network and an interstitial hydrogel portion within interstitial regions of the cellulose nanofiber network, the interstitial hydrogel portion comprising a hydrogel that is dry. The dry hydrogel implant may be inserted into the body and allowed to rehydrate in situ.

### CLAIM OF PRIORITY

This application claims priority to U.S. provisional patent application No. 63/408,087, filed on Sep. 19, 2022, titled “DRY HYDROGEL IMPLANTS”, which is herein incorporated by reference in its entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### BACKGROUND

Human cartilage has very unique properties. It is one of the few avascular tissues in the body. Cartilage is semipermeable and receives its nutrients from the synovial fluid which surrounds cartilaginous tissue in articulating joints, and which diffuses into the cartilage during motion of the joint. Cartilage itself also possesses viscoelastic and lubricating properties. Materials which are proposed for use in the repair or replacement of natural cartilage must possess physical and mechanical properties which are as close as possible to those of natural cartilage.

Historically the only choices available to patients with cartilage damage, especially the cartilage of an articulating joint, such as a knee or elbow, were to initially do nothing if the extent of the damage was only relatively minor in scope, which sooner or later usually led to a worsening of the condition and further damage to the cartilage and to the joint itself, with the patient feeling discomfort and pain when using the joint, thus ultimately requiring a complete joint replacement to restore mobility; or, if the extent of the damage was significant to start with, to immediately perform a complete joint replacement. In the case of very young patients, however, complete replacement of a joint is problematic in that the patient's overall skeletal bone structure is not yet fully developed and is still growing, so that the replaced joint may actually be outgrown and no longer be of appropriate size for the patient when their fully matured adult size, stature, and skeletal structure is attained. Moreover, in the past, many replacement knee, elbow joints and shoulder joints have typically had a maximum active useful life of only about ten years, due to wear and tear and erosion of the articulating surfaces of the joint with repetitive use over time, thereby necessitating periodic invasive surgery to replace the entire joint. For a very young patient this meant that they would have to face the prospect for several more such surgeries over their lifetime, notwithstanding progress and improvements in the wearability of materials used for joint surfaces that have been made and continue to be made as new materials are developed.

In recent years artificial cartilage materials have been proposed, and in particular, artificial cartilage made from hydrogels. Hydrogels are polymer networks typically swollen with water and are a promising synthetic material for replacement of cartilage because hydrogels can be made to have similar mechanical and tribological properties as natural cartilage. However, hydrogels may be difficult to use, and in particular may be difficult to implant without damage. Described herein are methods, hydrogel compositions, and apparatuses (e.g., implants) that may address these needs.

### SUMMARY OF THE DISCLOSURE

This disclosure relates generally to variations of artificial cartilage materials in implants suitable for repair of cartilage, including hydrogel composites and methods and for attaching a hydrogel composite to a surface of an implant in which the hydrogel is implanted dry and allowed to re-hydrate within the joint after implantation. Surprisingly, inserting the hydrogel material prior to hydration may prevent damage, and may allow sealing of the implantation site as the implant with the attached material is coupled to the tissue.

Described herein are method of processing hydrogel materials for use as artificial cartilage in implants, as well as method of implanting them. In particular, described herein are implants configured with a dried (e.g., un-hydrated) hydrogel. These hydrogels may be impregnated or infused in a nanofibrous material (e.g., a nanofiber network) and bound to a surface of an implant, such as a porous base, and then dried, so that the “dry” hydrogel material may be packaged and implanted into the patient and allowed to hydrate and swell following implantation.

The dried implant may have surface properties that prevent damage during implantation (e.g., making them hard and difficult to deform and/or scratch), while once implanted, the resulting hydrated hydrogel has physical properties, such as strength, modulus wear resistance, and coefficient of friction (COF) that approximates or exceeds that of healthy cartilage bound to bone.

The initial formation of the implant with the hydrogel may involve a strengthening process to increase the crystallinity and decrease the water content of the hydrogel, thereby improving its mechanical properties for implementation as cartilage replacement. As used described herein, strengthening of a hydrogel may include one or more steps of drying and annealing the hydrogel in communication with a fibrous support. The resulting dried hydrogel may be rehydrated within the body (e.g., by synovial fluid in the joint after the surgery is complete. Rehydration may therefore be performed after completion of the surgy (e.g., over hours and days) and may allow the implant to swell and seal around the implantation site, which may also held secure the implant in place, and prevent or reduce complications such as cysts caused by synovial fluid.

Approaches to creating synthetic cartilage by infiltrating a hydrogel into a nanofiber network for mimicking cartilage are described in International Patent Application No. PCT/US2021/040031, which is incorporated herein by reference in its entirety. The methods described herein may be used to form hydrogels that match or exceed the higher end of the range of strength of cartilage, while having a similar modulus, coefficient of friction, and resistance to wear of cartilage.

Described herein are method of making and using implants with dried hydrogel for mimicking or replacing cartilage. Any of the hydrogels described herein may be interdigitated with a nanofibrous network, such as a cellulose nanofiber network. The incorporated hydrogel, once hydrated, may have a crystalline structure that imparts high tensile and/or compressive strength to the hydrogel. In some examples, a reinforced hydrogel for use in an implant described herein may include a cross-linked cellulose nanofiber network; and a hydrogel infused within interstitial regions of the cross-linked cellulose nanofiber network, wherein the hydrogel has a crystallinity of 20% or greater. In some examples, the hydrogel comprises polyvinyl alcohol (PVA). In any of these examples the hydrogel may exclude (or substantially exclude) PAMPS. The hydrogel may be >90% PVA (e.g., >92%, >93%, >94%, >95%, >96%, >97%, >98%>99%, etc.) of PVA that has been annealed as described herein.

Described herein are implants comprising: an implant body and a dried, cellulose-reinforced hydrogel material comprising: a cross-linked cellulose nanofiber network bonded to the porous surface of the implant body by a cement; and a dried hydrogel impregnated in the cross-linked cellulose nanofiber network. The implant body may include a porous surface. For example, the implant body may be a titanium body with a bone-facing porous surface and a hydrogel-facing non-porous surface.

The hydrogels may be comprised of one or more polymers. In some examples, the hydrogel may include polyvinyl alcohol (PVA). In some cases, the hydrogel may include only one type of polymer. In some variations, the hydrogel is comprises of one or more of: polyvinyl alcohol (PVA), poly(2-acrylamido-2-methyl-1-propanesulfonic acid sodium salt (PAMPS), poly-(N,N′-dimethyl acrylamide) (PDMAAm), copolymers of 1-vinylimidazole and methacrylic acid, amphiphilic triblock copolymers, polyampholyte hydrogels, a PVA-tannic acid hydrogel, a poly(N-acryloyl) glycinamide hydrogel, polyacrylic acid-acrylamide-C18 hydrogel, Guanine-boric acid reinforced PDMAAm, polyelectrolyte hydrogels, a poly(acrylonitrile-co-1-vinylimidazole) hydrogel (e.g., a mineralized poly(acrylonitrile-co-1-vinylimidazole) hydrogel), a polyacrylic acid-Fe3+-chitosan hydrogel, a poly(methacrylic acid) gel, a Graphene oxide/Xonotlite reinforced polyacrylamide (PAAm) gel, a poly(stearyl methacrylate)-polyacrylic acid gel, an annealed PVA-polyacrylic acid hydrogel, supramolecular hydrogels from multiurea linkage segmented copolymers, polyacrylonitrile-PAAm hydrogel, a microsilica reinforced DMA gel, a Agar-polyhydroxyethylmethacrylate gel, a polyfacryloyloethyltrimethylammonium chloride hydrogel, a poly(3-(methylacryloylamino) propyl-trimethylammonium chloride hydrogel, a poly(sodium p-styrenbesulfonate) hydrogel, a polyethylene glycol diacrylate hydrogel, and a polyethylene glycol hydrogel. In some cases it may be beneficial to exclude PAMPS (e.g., having no PAMPS, having less than 0.1%, less than 0.5%, less than 1%, etc.).

The nanofiber network may comprise a cellulose nanofiber network. The nanofiber network may comprise a cross-linked cellulose nanofiber network. In some examples the nanofiber network comprises a bacterial cellulous (BC). Additionally or alternatively, the nanofiber network may comprise at least one of: electrospun polymer nanofibers, poly(vinyl alcohol) (PVA) nanofibers, aramid nanofibers, aramid-PVA nanofibers, wet-spun silk protein nanofiber, chemically crosslinked cellulose nanofiber, and polycaprolactone (PCL) fibers.

A cellulose-reinforced hydrogel may include: a cellulose nanofiber network; and a hydrogel impregnated in the cellulose nanofiber network, wherein the hydrogel may be dried after impregnation into the cellulose nanofiber network. The cellulose-reinforced hydrogel may comprise bacterial cellulose and/or a hydrogel comprising polyvinyl alcohol (PVA).

The cellulose-reinforced hydrogels described herein may be formed by: infiltrating a hydrogel in a cellulose nanofiber network to form the cellulose-reinforced hydrogel; and annealing the hydrogel to dry (e.g., to reduce the water content) and to increase a crystalline content of the hydrogel. Annealing the hydrogel may include heating the cellulose-reinforced hydrogel. Annealing the hydrogel may include heating the cellulose-reinforced hydrogel to decrease a water content of the hydrogel. In some examples, the cellulose-reinforced hydrogel may be heated to a temperature ranging from 90-140° C. The hydrogel implant may be left in the dried configuration and prevented from contacting fluid (e.g., water), until implantation. For example, the implant may be sealed in an air-tight and/or water-tight container (bag, package, etc.).

Also described herein are methods of implanting an implant that include a dried hydrogel. For example, any of the methods may include implanting the hydrogel into the body in the dry state and allowing the hydrogel to be rehydrated within the body following the implantation. In some examples fluid (e.g., saline, etc.) may be added to rehydrate the implant. Alternatively or additionally, the dried hydrogel may be allowed to rehydrate absorption of synovial fluid from the joint following the implantation. Rehydrating the hydrogel may involve increasing a water content of the hydrogel to at least 20 wt % (from 5% or less in the dehydrated or un-hydrated configuration). Rehydrating the cellulose-reinforced hydrogel may include rehydrating to 30% or more water, 35% or more water, 40% or more water, 45% or more water, 50% or more water, 55% or more water, 60% or more water, 65% or more water, 70% or more water, 75% or more water, 80% or more water, etc. (wt %).

When used for partial knee resurfacing, the implant may be configured to wear an opposing cartilage surface to an extent not significantly greater than the extent to which cartilage wears cartilage. A top bearing surface of the implant may have a coefficient of friction (COF) that is not statistically different from that of cartilage.

The implants described herein may be configured as a medical implant, and may include a tissue engaging portion (e.g., a bone engaging portion such as a rod, screen, nail, etc.).

The nanofiber network may be secured to the implant (e.g., to a porous surface of the implant) by any appropriate method. For example, the nanofiber network may be secured to the implant by a cement. In some examples the nanofiber network is not secured to the implant by a cement; for example, the nanofiber network may be secured as a sheet or other layer over the surface and held down by a clamp or otherwise.

The implant may be formed of any appropriate biocompatible material. For example, the surface of the implant body may be titanium. The surface of the implant body may be one or more of: a stainless steel alloy, a titanium alloy, a Co—Cr alloy, tantalum, gold, niobium, bone, Al oxide, Zr oxide, hydroxyapatite, Tricalcium phosphate, calcium sodium phosphosilicate, poly(methyl methacrylate), polyether ether ketone, polyethylene, polyamide, polyurethane, or polytetrafluoroethylene.

In general, the nanofiber network may be coupled to the top bearing surface of the implant. The cross-linked cellulose nanofiber network may be attached over the top load surface by clamping and/or by adhesive. For example, the nanofiber network may be bonded by cement to the top load surface; in some examples, the cement is not bonded to the hydrogel; the cement is only bonded to the nanofiber network. Alternatively, in some examples the nanofiber networks may be coupled to the implant, so that the nanofiber network, is secured over the top bearing surface without the use of a chemical adhesive, such as an epoxy. Instead, the nanofiber network may be secured over the top bearing surface by a clamp. For example, a clamp may secure the nanofiber network (e.g., one or more sheets of BC) over the top bearing surface around a periphery of the top bearing surface. Thus, in general, the use of an adhesive (such an epoxy) is optional.

Any appropriate implant may be used. The surface of the implant (e.g., top bearing surface, which may be equivalently referred to as simply the bearing surface) may be at least at the region to which the nanofiber network is attached over, may be titanium, stainless steel, etc. the bearing surface (e.g., top bearing surface) may be convex, flat, concave, or some mixture of these. For example, the surface of the implant body may comprise one or more of: a stainless steel alloy, a titanium alloy, a Co—Cr alloy, tantalum, gold, niobium, bone, Al oxide, Zr oxide, hydroxyapatite, Tricalcium phosphate, calcium sodium phosphosilicate, poly(methyl methacrylate), polyether ether ketone, polyethylene, polyamide, polyurethane, or polytetrafluoroethylene.

Also described herein are methods of making and/or using these implants. For example, described herein are methods of attaching a hydrogel to a surface. Any of these methods may include: infiltrating a hydrogel in a cellulose nanofiber network to form the cellulose-reinforced hydrogel; and annealing the hydrogel to increase a crystalline content of the hydrogel. For example, annealing the hydrogel may include heating the cellulose-reinforced hydrogel. In some examples, annealing the hydrogel may include heating the cellulose-reinforced hydrogel to decrease a water content of the hydrogel. For example, the cellulose-reinforced hydrogel may be heated to a temperature ranging from 90-140° C.

In the dry (un-hydrated) configuration, the outer surface of the hydrogel may be smooth (e.g., may have a roughness of less than 30 microns). The dry outer surface may be mechanically polished to a roughness of less than 30 microns. In some cases, the outer surface may be formed smooth by molding, including molding the heated polymer using a smooth mold. For example, infiltrating the nanofiber network with hydrogel may include molding the hydrogel so that an outer surface of the hydrogel has a roughness of less than 30 microns. Molding the outer surface may also allow a manufacturer to form the outer surface into any desired shape. For example, the shape may be concave, convex, saddle shaped, etc. Any desired shape (and smoothness) may be formed, e.g., by molding and/or polishing.

In any of these methods securing the (e.g., dry) nanofiber network may include securing, such as clamping and/or cementing, a freeze-dried nanofiber network. As mentioned above, any of these devices and methods may use a dry nanofiber network that comprises a cellulose nanofiber network. The dry nanofiber network may comprise at least one of: electrospun polymer nanofibers, poly(vinyl alcohol) (PVA) nanofibers, aramid nanofibers, Aramid-PVA nanofibers, wet-spun silk protein nanofiber, chemically crosslinked cellulose nanofiber, or polycaprolactone (PCL) fibers.

For example, described herein are implants, comprising: an implant body having a top bearing surface; an anchoring base (which may extend from a back of the top bearing surface); and a dry cellulose-reinforced hydrogel comprising: a cross-linked cellulose nanofiber network secured over the top bearing surface of the implant body; and an interstitial hydrogel portion within interstitial regions of the cross-linked cellulose nanofiber network, wherein the interstitial hydrogel portion has a crystallinity of 20% or greater. The interstitial hydrogel portion may polyvinyl alcohol (PVA). The cellulose-reinforced hydrogel may comprise less than 20% by weight of water. The cross-linked cellulose nanofiber network may comprise bacterial cellulose (BC). The cross-linked cellulose nanofiber network may be secured over the top bearing surface by a clamp. In some examples the cross-linked cellulose nanofiber network comprises one or more sheets of bacterial cellulous (BC) held over the top bearing surface by a clamp secured to a lip or rim of the top bearing surface. The clamp may be used to secure the cross-linked cellulose nanofiber network without the need for epoxy. Alternatively any of these implants may include an adhesive.

For example, described herein are implants (e.g., in some examples resurfacing implants), comprising: an implant body having a bearing surface; an anchoring base coupled to the implant body; and a cellulose-reinforced dry hydrogel comprising: a cross-linked cellulose nanofiber network secured over the bearing surface of the implant body; and an interstitial hydrogel portion within interstitial regions of the cross-linked cellulose nanofiber network, wherein the interstitial hydrogel portion has a water content of 20% or less. The water content of the cellulose-reinforced dry hydrogel including the interstitial hydrogel portion may be less than 20% (e.g., less than 19%, less than 18%, less than 17%, less than 16%, less than 15%, less than 14%, less than 13%, less than 12%, less than 11%, less than 10%, less than 9%, less than 8%, etc.). In some examples the water content of the cellulose-reinforced dry hydrogel including the interstitial hydrogel portion is less than 5%.

The interstitial hydrogel portion may comprise, in particular, polyvinyl alcohol (PVA). The cross-linked cellulose nanofiber network may comprise bacterial cellulose. For example, the cross-linked cellulose nanofiber network may comprise one or more sheets of bacterial cellulous (BC) held over the top bearing surface by a clamp secured to a lip or rim of the top bearing surface. The cross-linked cellulose nanofiber network may be secured over the top bearing surface by a clamp. In any of these examples, the cross-linked cellulose nanofiber network is not cemented to the bearing surface.

The interstitial hydrogel portion (e.g., in some examples, the PVA) may have a crystallinity of 20% or greater (e.g., 22% or more, 24% or more, 25% or more, 26% or more, 28% or more, 30% or more, 32% or more, 34% or more, 35% or more, 36% or more, 38% or more, 40% or more, 42% or more, 44% or more, 45% or more, 46% or more, 48% or more, 50% or more, etc.).

For example, an implant may include: an implant body having a bearing surface; an anchoring base coupled to the implant body; and a cellulose-reinforced dry hydrogel comprising: a cross-linked cellulose nanofiber network secured over the bearing surface of the implant body; and an interstitial polyvinyl alcohol (PVA) hydrogel material within interstitial regions of the cross-linked cellulose nanofiber network, wherein the water content of the cellulose-reinforced dry hydrogel including the interstitial hydrogel portion is 20% or less.

Any of the methods described herein may be methods of forming an implant having a cellulose-reinforced hydrogel, and may include: attaching a cross-linked cellulose nanofiber network to a top bearing surface of the implant; infiltrating a hydrogel material within interstitial regions of the cross-linked cellulose nanofiber network to form the cellulose-reinforced hydrogel; and heating the cellulose-reinforced hydrogel so that a water content of the cellulose-reinforced hydrogel is 20% or less. Heating may comprise heating the cellulose-reinforced hydrogel so that the water content of the cellulose-reinforced hydrogel is 11% or less. In some examples heating comprises heating the cellulose-reinforced hydrogel so that the water content of the cellulose-reinforced hydrogel is 5% or less. The hydrogel material may include polyvinyl alcohol (PVA). The cellulose-reinforced hydrogel may be heated to a temperature ranging from 90-140° C.

Any of the implants described herein may be implants for a knee resurfacing. For example, an implants for a knee resurfacing may include: a top bearing surface comprising a cellulose-reinforced hydrogel comprising: a cellulose nanofiber network; and a polyvinyl alcohol (PVA) hydrogel material impregnated in the cellulose nanofiber network to form a cellulose-reinforced hydrogel, wherein the cellulose-reinforced hydrogel has a water content of less than 20%. The cellulose-reinforced hydrogel may have a water content of less than 10%, less than 5%, etc. As mentioned, the PVA material may have a crystallinity of 20% or greater.

Also described herein are methods of implanting a resurfacing implant, the method may include: forming an opening in a bone (e.g., by drilling, tamping, etc.); inserting the resurfacing implant into the opening in the bone so that a bearing surface of the resurfacing implant faces away from the bone, wherein the bearing surface comprises a cellulose-reinforced hydrogel having a water content of 20% or less, wherein the cellulose-reinforced hydrogel comprises a cross-linked cellulose nanofiber network that is impregnated with a polyvinyl alcohol (PVA) hydrogel; and allowing the cellulose-reinforced hydrogel to rehydrate in situ to have a water content of greater than 30%. The bearing surface may be proud or flush with the bone surface. Allowing the cellulose-reinforced hydrogel to rehydrate may include swelling the resurfacing implant to seal an edge of the resurfacing implant adjacent to the bone. Any of these methods may include dehydrating the resurfacing implant prior to inserting. The cellulose-reinforced hydrogel may have a crystallinity of 20% or more.

In general, the methods and apparatuses described herein may be used with any of the methods, apparatuses and compositions described in International Patent Application No. PCT/US2021/040031, titled “NANOFIBER REINFORCEMENT OF ATTACHED HYDROGELS”, filed on Jul. 1, 2021, which is herein incorporated by reference in its entirety.

All of the methods and apparatuses described herein, in any combination, are herein contemplated and can be used to achieve the benefits as described herein.

### BRIEF DESCRIPTION OF THE DRAWINGS

A better understanding of the features and advantages of the methods and apparatuses described herein will be obtained by reference to the following detailed description that sets forth illustrative embodiments, and the accompanying drawings.

FIG. 1A is an illustration of an exemplary process for attachment of a hydrogel to a porous base by a Nanofiber-Enhanced STicking (NEST) method. In this example, a nanofibrous sheet (e.g., bacterial cellulose) is attached to a surface (e.g., a porous base such as porous titanium) with an adhesive (e.g. α-TCP cement), after which the hydrogel components are infiltrated into the nanofibrous sheet.

FIG. 1B shows an example of a hydrogel bonded to a titanium plug.

FIG. 1C shows an SEM image of a surface of an exemplary freeze-dried bacterial cellulose sheet.

FIGS. 2A and 2B schematically illustrate examples of implants including a hydrogel attached (e.g., forming the surface) as described herein.

FIG. 3A is an image illustrating one example of a method of attaching a hydrogel to a metallic plug, including using a clamp (e.g., a shape memory alloy clamp).

FIGS. 3B and 3C illustrate examples of a fixture that may be used for aligning forming the materials described herein (e.g., aligning the BC, including a rod, cut BC, and ring clamp) as described herein: FIG. 3B shows a perspective view of the fixture; and FIG. 3C shows a sectional view through the fixture.

FIG. 3D is an image showing an exemplary sheet of bacterial cellulose (BC) cut (e.g., with legs or crenellations) for wrapping over the edge of a bearing surface (e.g., metal rod, head, etc.).

FIG. 4 shows an example of a process for attaching the BC-PVA-PAMPS hydrogel to a titanium implant for treatment of osteochondral defects.

FIG. 5A shows an example of an implant including a hydrated hydrogel infused within a fibrous (bacterial cellulose) network.

FIG. 5B shows the implant of FIG. 5A after tamping with an edge of a tamp, illustrating a potential source of damage to the hydrogel.

FIG. 6A shows an example of an implant including a dried hydrogel infused within a fibrous (bacterial cellulose) network.

FIG. 6B shows the implant of FIG. 6A after tamping with an edge of a tamp in the same manner (force and location) as FIG. 5B, showing no visible damage to the hydrogel.

FIGS. 7A and 7B illustrate implantation of a dry hydrogel implant into a knee joint, showing swelling. FIG. 7A shows the implant inserted at day 0. FIG. 7B shows the same implant after one day within the joint.

FIGS. 8A and 8B show another example of a dry hydrogel implant into a knee joint, showing swelling. FIG. 8A shows the implant inserted at day 0. FIG. 8B shows the same implant after one day within the joint.

FIG. 9 is a chart illustrating one method if implanting a dry hydrogel implant as described herein.

### DETAILED DESCRIPTION

Described herein are dry hydrogel (“dried hydrogel,” “un-hydrated hydrogel” or “dehydrated hydrogel”) compositions for the long-term repair of cartilage. Specifically, described herein are methods and apparatuses for implanting dry hydrogels that re-hydrate within the body to form a crystalline structure that impart tensile and compressive strengths to the hydrogels that equal or exceed that of cartilage. The dried hydrogels may be incorporated in a nanofiber network (e.g., cellulose, such as bacterial cellulose) to facilitate ease of implantation and attachment within a patient's body. These apparatuses may be robustly implanted and may seal the implantation site and are thus well-suited for implementation on knee implants.

The implants described herein include a hydrogel that is infused, impregnated or interdigitated within the fibrous network, where the fibrous network is attached to an implant surface (e.g., by chemical and/or mechanical attachment, such as a clamp). The hydrogel is dehydrated (e.g., annealed), so that it has less than 20% (e.g., less than 19%, less than 18%, less than 17%, less than 16%, less than 15%, less than 14%, less than 13%, less than 12%, less than 11%, less than 10%, less than 9%, less than 8%, less than 7%, less than 6% less than 5%, etc.) water in the dried state. The resulting dried hydrogel surface (and fibrous network) is much stronger than in the hydrated state and can be applied into the target location of the body, which may involve tamping or otherwise inserting into bone, without risk of damaging the hydrogel/fibrous network. In particular, these dried hydrogel networks are dry and strong upon implantation, so that it may be tamped in to achieve a strong press fit, but hydrates within the body within about 24 hours, becoming a lubricious hydrogel with cartilage-equivalent coefficient of friction.

For example, the implants described herein may be formed of a hydrogel (e.g., including but not limited to PVA), a fibrous network (e.g., cellulose, such as bacterial cellulose), and may be affixed to a surface of an implant (having a stem for inserting into bone and a wider outer facing surface onto which the fibrous network and hydrogel may be attached. In some examples the dried hydrogel implants may be assembled by attaching the fibrous network (e.g., a sheet of bacterial cellulose) to the outer facing surface. After assembling the cellulose onto the implant, the implant is heated in a mixture of the PVA and water for 24 hours to infuse the molten PVA into the cellulose. The molten PVA is then molded to be 1.2 mm thick. It is then dried in an oven at 90 C for 24 hours, whereupon the hydrogel shrinks to be 0.85 mm thick. This is the “dry” state.

The implant may be stored in the dry configuration, e.g., by sealing within packaging that prevents exposure to water, For example, the implant may be packaged in a sealed contained that is air-tight and/or moisture-tight. Before implantation, the implant may be removed from the packaging and applied to the patient directly, without the need to rehydrate first. In the dry state the implant may be driven into the bone using a tamp of other tool, with little risk of damage to the surface of the implant. Once implanted, the dry hydrogel may rehydrate within the body. Although fluid (saline, ringers, blood, etc.) may be applied during and immediately after the procedure, in general no additional fluid is necessary. The implant will rehydrate itself within about 24 hours or less (e.g., from synovial fluid) and may regain the hydrated thickness, e.g., of about 0.35 mm.

In implanting implant including a dry hydrogel it may be beneficial to recess the implant, so that in the hydrated configuration the implant may be flushed or very slightly proud of the implantation site. In addition, the hydration of the dry hydrogel may help seal the hydrogel of the implant around the edge of the implantation site. This may prevent synovial fluid from getting into the bone and developing cysts.

Although the methods and apparatuses described herein are primary described in the context of PVA, other hydrogels may be used. Previously methods of forming implants with hydrogels comprising a bacterial cellulose (BC) network infused with both polyvinyl alcohol (PVA) and poly(2-acrylamido-2-methyl-1-propanesulfonic acid sodium salt (PAMPS), referred to as BC-PVA-PAMPS hydrogels, are described in International Patent Application No. PCT/US2021/040031, which is incorporated herein by reference in its entirety.

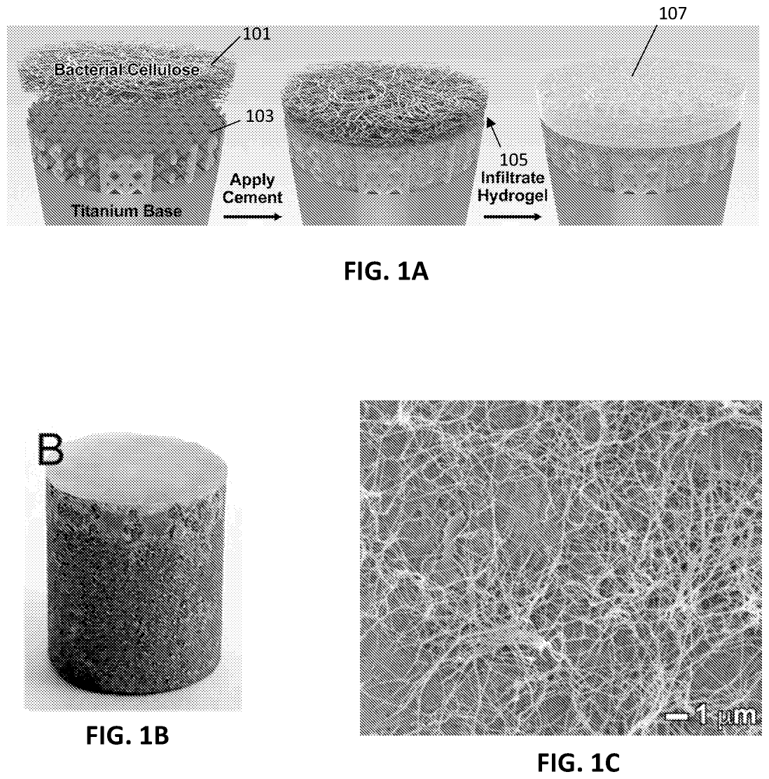

FIGS. 1A-1C illustrate an example of an apparatus in which a hydrogel has been bonded to an implant surface as described herein. The hydrogel may be coupled to the implant surface by first attaching a layer of nanofibrous material, such as cellulous (e.g., bacterial cellulous) to an implant base using an adhesive (e.g., cement). The nanofibrous material may be dry (e.g., before attaching to the implant base). The attaching surface of the implant base may be porous, for example, to enhance adhesion. The nanofibrous layer may then be infiltrated with a hydrogel component (e.g., material). In this way, the nanofibrous portion may be secured with an adhesive (e.g., cement) that can penetrate and secure the porous bacterial cellulose network to the surface and may create an interdigitating bond without the interference of water. Once the hydrogel material is infiltrated within the nanofibrous network, the reinforced hydrogel may be processed (e.g., annealed) to dehydrate the hydrogel as described herein.

For example, in FIG. 1A, the nanofibrous portion is bacterial cellulose (BC) 101 that is applied to the prepared surface of the implant (shown in this example as a titanium base, having pores) 103. A cement (e.g., any appropriate medical or dental grade cement may be used) is applied and secures the dry bacterial cellulose to the implant surface. Thereafter the hydrogel material 105 may be infiltrated into the nanofibrous portion, resulting in the complete hydrogel 107 attached to the base 103 via the bacterial cellulose 101. The reinforced hydrogel 107 then undergoes a crystal restructuring process to enhance its mechanical properties.

FIG. 1B shows an example of a titanium implant (e.g., plug) to which a cellulous-reinforced hydrogel has been attached, as described herein. In this example, the nanofibrous portion (e.g., BC) of the hydrogel is bonded via an adhesive to the porous surface of the implant, and the hydrogel is linked to the nanofibrous portion. Any appropriate adhesive (e.g., cement) may be used to adhere the nanofibrous portion of the hydrogel to the surface of the implant. In some variation the cement is α-tricalcium phosphate (α-TCP), a hydroxyapatite-forming cement that may be used for attachment of the hydrogel due to its biocompatibility, osteoconductivity, and shear strength, which may exceed that of cyanoacrylate. In some cases, α-TCP is combined with phosphoserine (PPS) to promote adhesion. In some cases, the hydroxyapatite is reinforced with stainless-steel powder (SSP) (e.g., with an average particle size of 150 μm) to hinder crack propagation. As will be described in greater detail below, in some examples an adhesive is not used, and the nanofiberous portion is secured to the bearing surface by a mechanical means (such as a clamp).

As described herein, the nanofibrous portion (e.g., BC) may be treated to dried (e.g., freeze-dried) to increase adhesion to the nanofibers. FIG. 1C is a scanning electron microscope (SEM) image of the surface of an exemplary freeze-dried piece of BC, which shows that it consists of many nanoscale fibers that present a large surface area for attachment with an adhesive. In some examples, multiple freeze-thaw cycles are performed, which may increase tensile strength (once the hydrogel is infused therein) and/or increase the shear strength of the adhesion of the reinforced hydrogel to the implant base.

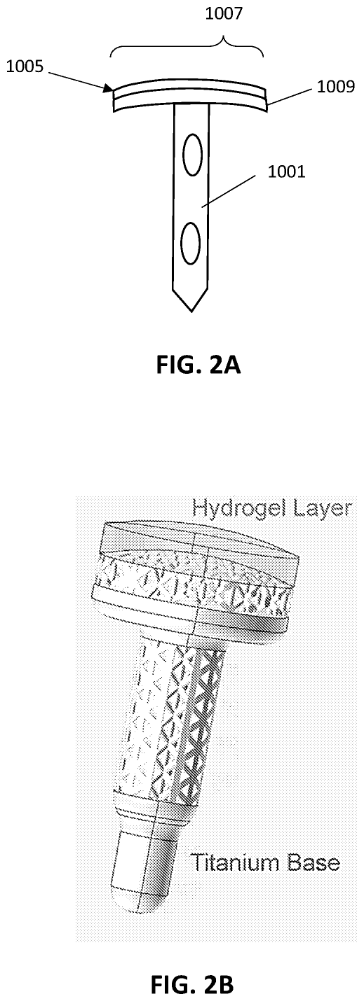

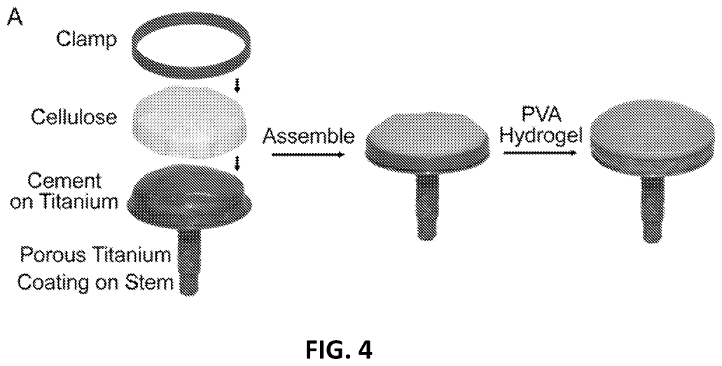

Any appropriate implant may include a hydrogel. FIGS. 2A-2B illustrate two examples of implants configured as nail-or tack-like structures that may be inserted into a bone to replace or repair a defect in cartilage, e.g., for partial knee resurfacing. An implant for partial knee resurfacing may be relatively large and may be curved to mimic the natural curvature of the femoral condyle. FIG. 4 shows an image of an implant 20 mm in diameter with a radius of curvature of 20 mm. An implant diameter of 20 mm is a typical size used for an osteochondral allograft, and a 20 mm radius of curvature is within the range of typical curvatures for the femoral condyle. A 0.25-mm-thick coating of commercially pure titanium was applied to the stem of the implant and underneath the base with a plasma spray process in order to improve integration with bone. Such an implant, with a dried hydrogel as described herein, may be used to resurface the knee. The surgeon may drill out a hole over the defect site that is complementary to the shape of the hydrogel-capped implant. The hole may be drilled to the same depth or slightly deeper than the dried hydrogel top of the implant. The dry hydrogel-capped implant may then be pressed or tamped into the hole to replace the damaged cartilage, while in the dry state. The outer surface of the dry hydrogel may be slightly recessed upon implantation and may swell to fill (or extend slightly out of) the hole.

As used herein, an implant may have any appropriate structure for implanting into a body. In some (non-limiting) examples, the implants may have a shape that allows them to be implanted into bone, with a hydrogel attached to an outward-facing side. For example, FIGS. 2A and 2B illustrate examples of implants to which a hydrogel has been attached, as described herein. In FIG. 2A, the implant includes a base 1001 (e.g., a titanium base) having an elongate pin-shape that may be, for example, 2 mm×7 mm (tapering to about 1.5 mm at about 3 mm from the end). The base may include one or more channels, openings, passages, etc. for ingrowth of bone. The implant also includes a top portion 1005 that may be curved (e.g., with a single curvature or a double-curvature. For example, the surface may be curved with a radius of curvature of about 17 mm (single curvature) or about 19 mm×12 mm (double curvature). In FIG. 2A the top is approximately 7 mm in diameter 1007. The outer surface of the implant may be approximately 1 mm thick or thicker 1009 and may be about 70% porous, or greater. The hydrogel may be attached to the top surface. The hydrogel in this example is a triple-network hydrogel of BC-PVA-PAMPS and the BC is cemented to the porous top, while the PVA-PAMPS is impregnated into the BC. FIG. 2B shows a similar implant to that shown in FIG. 2A, in which a hydrogel is attached (e.g., via cementing the nanofibrous portion of the hydrogel to the porous surface of the implant, as shown. The implant in FIG. 2B is titanium.

As mentioned above, any of these implant surfaces may include a porous structure. The porosity of the implant surface may be, e.g., between 10% porous and 90% porous, e.g., between 30% porous and 90% porous, between 55% porous and 95% porous, between 65% porous and 85% porous, etc.). The depth of the pores may also be varied. For example, the surface may be porous to a depth of between 0.1 mm and 5 mm, between 0.2 mm to 3 mm, between 0.5 mm to 2 mm (e.g., 0.2 mm or greater, 0.3 mm or greater, 0.5 mm or greater, 0.75 mm or greater, 1 mm or greater, 1.5 mm or greater, etc.).

As mentioned, any appropriate nanofibrous network may be used, including, but not limited to nanofibrous bacterial cellulose. Other nanofibrous networks may include electrospun polymer nanofibers such as poly(vinyl alcohol) (PVA) nanofibers, aramid nanofibers (e.g., Aramid-PVA nanofibers), wet-spun silk protein nanofiber, chemically crosslinked cellulose nanofiber, or polycaprolactone fibers (e.g., 3D woven PCL fibers). In addition, any appropriate double network hydrogels may be used, including but not limited to PVA and PAMPS. For example, other hydrogel-forming polymers may include poly-(N,N′-dimethyl acrylamide) (PDMAAm), copolymers of 1-vinylimidazole and methacrylic acid, double-network hydrogels based on amphiphilic triblock copolymers, polyampholyte hydrogels, a PVA-tannic acid hydrogel, a poly(N-acryloyl) glycinamide hydrogel, polyacrylic acid-acrylamide-C18 hydrogel, Guanine-boric acid reinforced PDMAAm, polyelectrolyte hydrogels, a poly(acrylonitrile-co-1-vinylimidazole) hydrogel (e.g., a mineralized poly(acrylonitrile-co-1-vinylimidazole) hydrogel), a polyacrylic acid-Fe3+-chitosan hydrogel, a poly(methacrylic acid) gel, a Graphene oxide/Xonotlite reinforced polyacrylamide (PAAm) gel, a poly(stearyl methacrylate)-polyacrylic acid gel, an annealed PVA-polyacrylic acid hydrogel, supramolecular hydrogels from multiurea linkage segmented copolymers, polyacrylonitrile-PAAm hydrogel, a microsilica reinforced DMA gel, a Agar-polyhydroxyethylmethacrylate gel, a polyfacryloyloethyltrimethylammonium chloride hydrogel, a poly(3-(methylacryloylamino) propyl-trimethylammonium chloride hydrogel, a poly(sodium p-styrenbesulfonate) hydrogel, a polyethylene glycol diacrylate hydrogel, a polyethylene glycol hydrogel, or hydrogels composed of a combination of these polymers.

The implants described herein may be formed of any appropriate material, including, but not limited to titanium and stainless steel. For example, a hydrogel may be attached as described herein to an implant surface (e.g., base, including a porous base) that is formed of a stainless steel alloy, other titanium alloys, Co—Cr alloys, tantalum, gold, niobium, bone, Al oxide, Zr oxide, hydroxyapatite, tricalcium phosphate, calcium sodium phosphosilicate (Bio glass), poly(methyl methacrylate), polyether ether ketone, polyethylene, polyamide, polyurethane, polytetrafluoroethylene, or other materials used for making implants.

Any of the implants described herein may include a hydrogel having a surface that is substantial smooth and/or is shaped in a predetermined configuration, such as (but not limited to) concave, convex, saddle-shaped, etc. For example, any of these apparatuses (e.g., implants) may have a surface roughness that is less than 30 microns. In some cases, the surface may be formed smooth by molding. In some cases, the surface may be formed smooth by polishing or sanding. For example, once the additional hydrogel materials have formed the network (e.g., the nanofibrous-reinforced network), the hydrogel coating may optionally be finished by polishing; in particular, the surface may be sanded to polish to a roughness of less than 30 microns. Polishing may be performed by sanding (e.g., using a fine grit sanding surface, such as a 600, 400, 320, etc. grit).

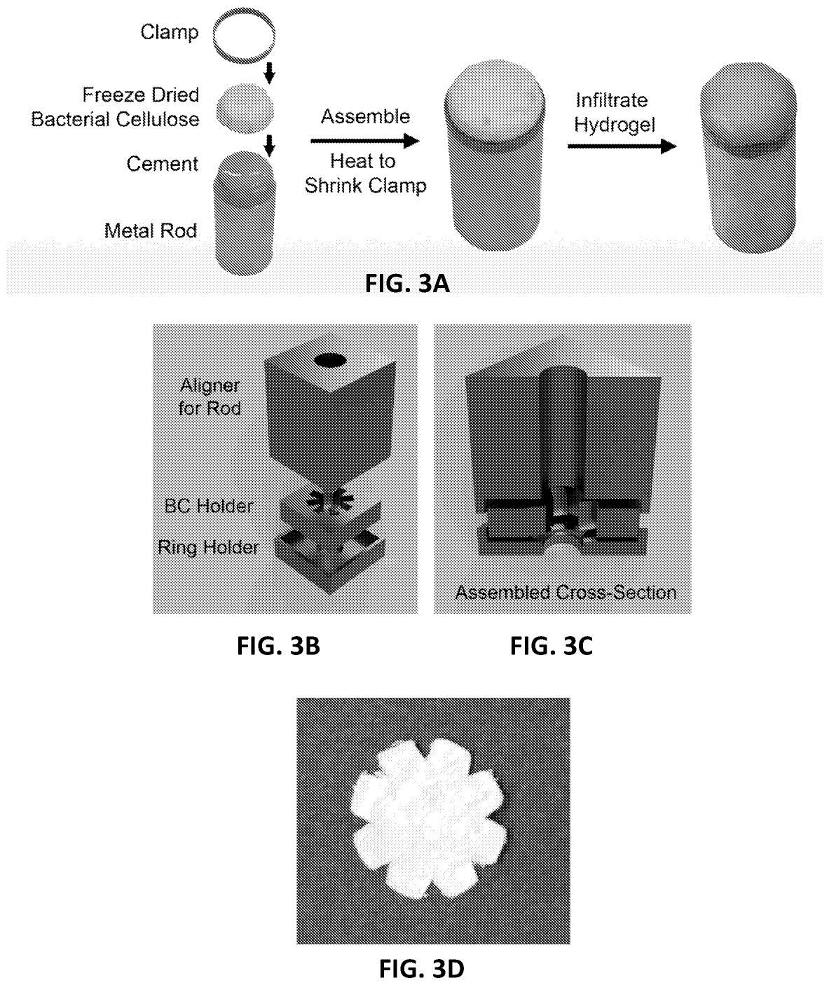

FIGS. 3A-3C illustrate a brief overview of an example of how the hydrogel may be attached to a metal base (e.g., of the top bearing surface). In this example freeze-dried BC sheets were cut into octagonal shapes with 8 projections (e.g., “legs”) that can be bent over the edges of the implant, as shown in the example of FIG. 3D. This cut may remove excess BC that would otherwise be folded up on the sides of the cylinder. The pieces of cut BC were then placed into a fixture that facilitates centering and alignment of the ring clamp with the pieces of BC and the metal rod. The metal rod was pushed down through the fixture so that the ring pushed the pieces of BC onto the metal rod. This process of pushing the ring over the BC and onto the rod could also be done by hand. The use of an alignment features, such as shown in FIGS. 3B-3C may help consistently center the pieces during assembly. The sample may then be clamped, e.g., by heated in an oven at 90° C. to initiate clamping in a shape-memory alloy material preset as described herein (which starts at a temperature of 50° C.). The part was then heated in a hydrothermal bomb at 120° C. for 24 hours with PVA to infiltrate the polymer into the BC. The part was then dried, as described herein and stored dry.

### EXAMPLES

In general, the density of bone and the stiffness of the bone is highly variable. The density and stiffness of bone may vary by 10× (0.14-1.4 g/cm³) or more. When inserting an implant such those illustrated above, a thumb press fit is generally recommended, but it may be particularly challenging, as it has to be strong enough for soft bone but not too hard to push in for hard bone. Preliminary data testing the force needed to apply a thumb press fit into bone having a modulus that may vary from 58-445 MPa showed that for an implant that is nominally 5.5 mm, the actual machined diameter range will be 5.4-5.6 mm (100 μm tolerance). The size ranges of the implant and the size of the hole into which it may be inserted by a press fit is very narrow. For example, for a 5.6 mm implant, the hole size must be between 5.4-5.5 mm to achieve a sufficient, but not too much, press fit in nominal bone. For a 5.4 mm implant, the hole size must be 5.2-5.3 mm to achieve the same “just right” press fit. Further, these ranges do not overlap. Thus an adequate press fit with a single drill step is not possible. Some adaptation to the surgery will likely be necessary, increasing the complexity, time and cost of the procedure.

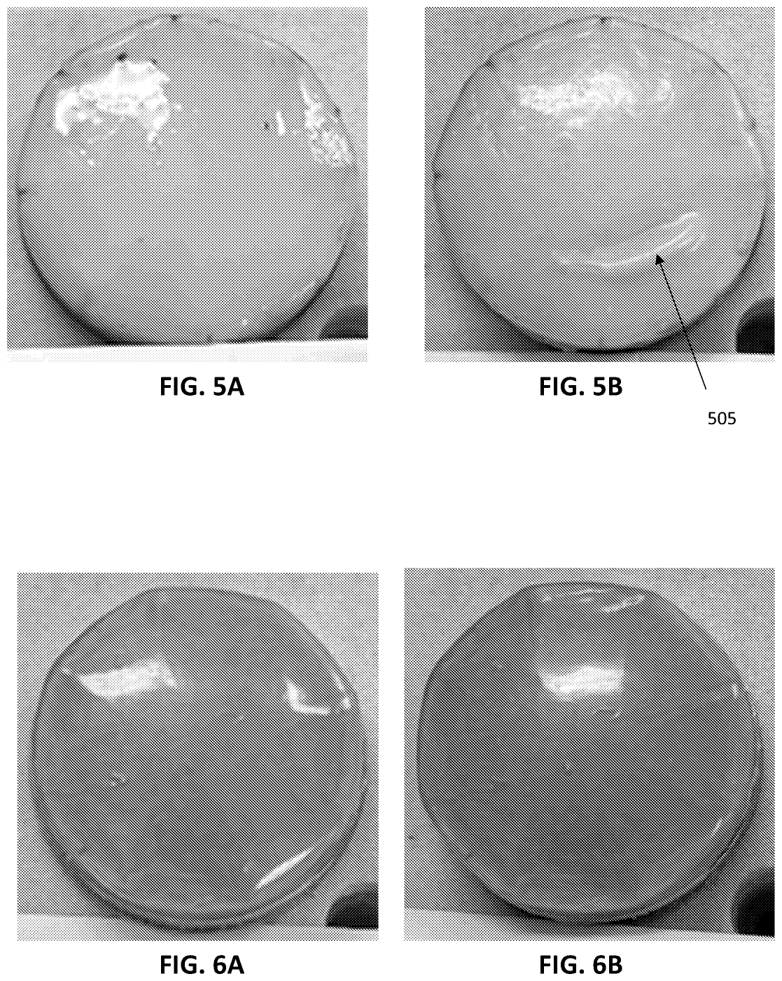

As a result, it is often necessary to provide additional tools (e.g., tamps, presses, etc.) to apply implant into the body. However, there is a strong chance that implantation may damage the hydrogel, e.g., when a tamp or other tool is used, particularly where the hydrogel covers the majority, if not all, of the outer surface of the hydrogel. For example, FIGS. 5A and 5B illustrate a potential issue with inserting hydrated hydrogel implants. In FIG. 5A a top view of a hydrated hydrogel implant (similar to those shown in FIGS. 2A-2B and 4) is shown prior to implantation. The hydrated hydrogel is easily damaged by the edge of the tamp, as shown in FIG. 5B., which shows the same hydrogel as in FIG. 5A. A damaged region 505 is visible; this damage may result in problems following insertion, as the damaged surface may provide a biological response, and lead to post-operative pain.

In contrast the use of a dried hydrogel prevents and protects against such damage. For example, FIG. 6A illustrates a top view of a dry hydrogel implant. The dry hydrogel implant has a hardened outer surface (akin to plastic) that may hydrate in vivo, but until then may resist damage. For example, the same implant as shown in FIG. 6A is shown in FIG. 6B after hitting with a tamp edge in a similar manner (same force and location) as in FIGS. 5A and 5B. As shown, no visible damage is present in FIG. 6B. In general, the implant may be driven into the bone vigorously with a tamp or other tool, with little risk of damage to the dried hydrogel. Once implanted, the hydrogel will swell and hydrate. Thus, instead of relying on a press fit using just the surgeon's fingers (which may be difficult if not impossible to do accurately and consistently), a tamp or other force-applying tool may be used with a dry hydrogel. When implanted as a dry hydrogel, the implant may starts recessed instead of flush to ensure correct implantation depth is achieved. However the use of a dry implant may help ensure that a firm press fit is used without damaging the implant.

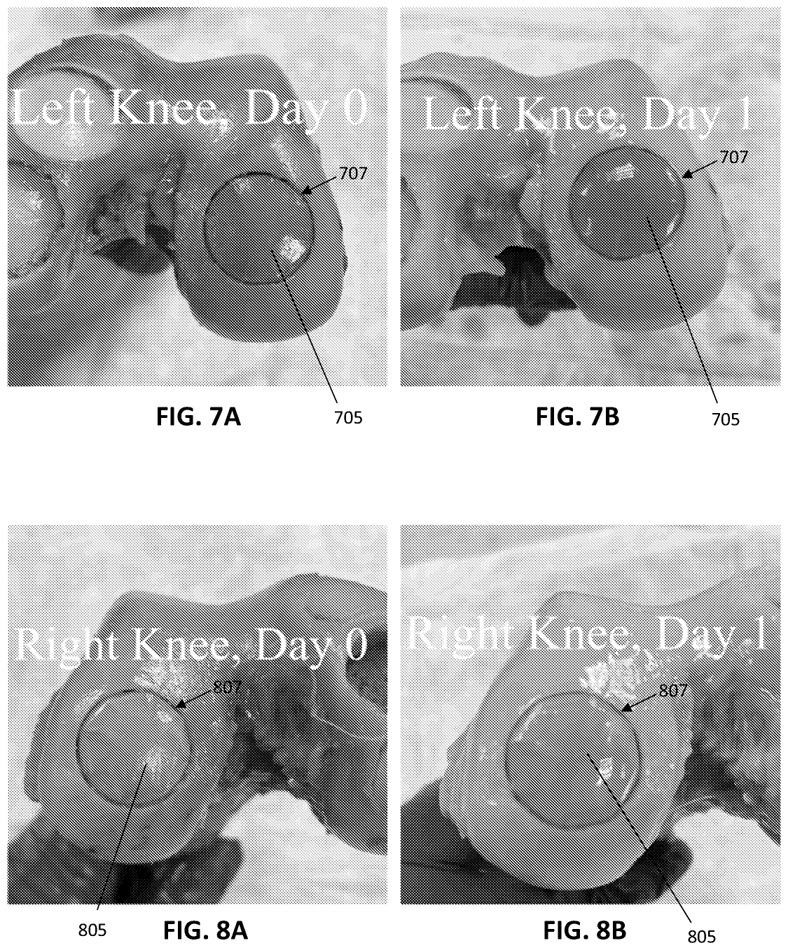

In practice, the use of a dry hydrogel implant may be easier and more effectively than expected. As mentioned above, the dry hydrogel implant will rehydrate due to the synovial fluid within about 24 hours or less. Because the hydrogel region swells, the drilled holes for the implant may be slightly larger than previously used. For example, FIGS. 7A and 7B illustrate a first example of a dry implant 705 inserted into a hole drilled into a bone (left knee), immediately following implantation. After 24 hours of rest, the same knee was exposed, showing (in FIG. 7B) that the hydrogel has hydrated and expanded, swelling within 24 hours. The space between the cartilage and implant 707 got smaller. The hydrogel may be made wider so that is swells and completely seals around the edge, which may prevent the synovial fluid from getting into the bone and causing cysts.

FIGS. 8A and 8B shows similar results. In FIG. 8A the dry hydrogel implant 805 is inserted into a slightly larger opening, while in FIG. 8B (after 25 hours) the hydrogel on the implant 805 has hydrated and swelled so that, again it has expanded against the opening 807, as shown.

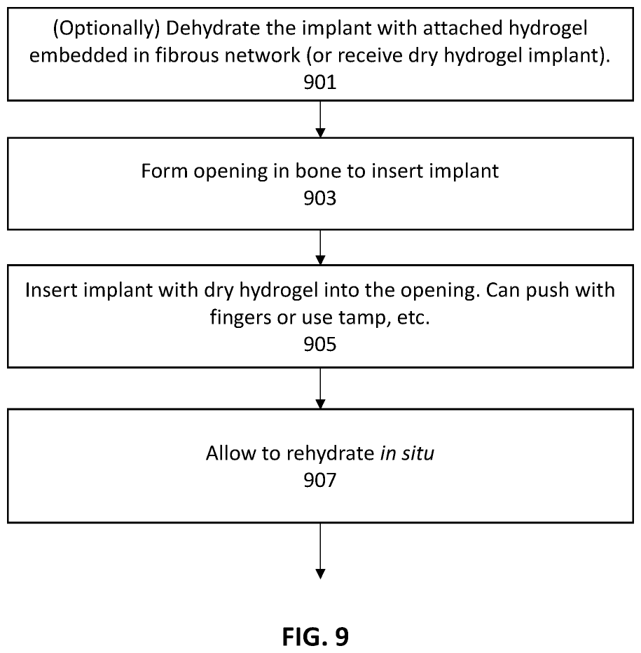

For example, a method of implanting a hydrogel-containing implant is shown in FIG. 9. In this example, the implant may be received in the dry configuration (e.g., dry hydrogel implant) or it may be dried as described herein 901 (e.g., heating at an elevated temperature for >1 hour or until the percentage of water is less than, e.g., 20%. The body region may be prepared by, e.g., forming the opening (e.g., by drilling) in the bone to insert the implant 903. Thereafter the dry hydrogel implant may be inserted 905. A tamp or other force-applying tool may be used. The hydrogel may then be allowed to rehydrate within the body (e.g., over 24 hours or less) 907.

Any of the methods (including user interfaces) described herein may be implemented as software, hardware or firmware, and may be described as a non-transitory computer-readable storage medium storing a set of instructions capable of being executed by a processor (e.g., computer, tablet, smartphone, etc.), that when executed by the processor causes the processor to control perform any of the steps, including but not limited to: displaying, communicating with the user, analyzing, modifying parameters (including timing, frequency, intensity, etc.), determining, alerting, or the like.

It should be appreciated that all combinations of the foregoing concepts and additional concepts discussed in greater detail below (provided such concepts are not mutually inconsistent) are contemplated as being part of the inventive subject matter disclosed herein and may be used to achieve the benefits described herein.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise. Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

In general, any of the apparatuses and methods described herein should be understood to be inclusive, but all or a sub-set of the components and/or steps may alternatively be exclusive and may be expressed as “consisting of” or alternatively “consisting essentially of” the various components, steps, sub-components or sub-steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

1. An implant, comprising:
an implant body having a bearing surface;
an anchoring base coupled to the implant body; and
a cellulose-reinforced dry hydrogel comprising:
a cross-linked cellulose nanofiber network secured over the bearing surface of the implant body; and
an interstitial hydrogel portion within interstitial regions of the cross-linked cellulose nanofiber network, wherein the interstitial hydrogel portion has a water content of 20% or less.

2. The implant of claim 1, wherein the water content of the cellulose-reinforced dry hydrogel including the interstitial hydrogel portion is less than 10%.

3. The implant of claim 1, wherein the water content of the cellulose-reinforced dry hydrogel including the interstitial hydrogel portion is less than 5%.

4. The implant of claim 1, wherein the interstitial hydrogel portion comprises polyvinyl alcohol (PVA).

5. The implant of claim 1, wherein the cross-linked cellulose nanofiber network comprises bacterial cellulose.

6. The implant of claim 1, wherein the cross-linked cellulose nanofiber network is secured over the top bearing surface by a clamp.

7. The implant of claim 6, wherein the cross-linked cellulose nanofiber network comprises one or more sheets of bacterial cellulous (BC) held over the top bearing surface by a clamp secured to a lip or rim of the top bearing surface.

8. The implant of claim 1, wherein the cross-linked cellulose nanofiber network is not cemented to the bearing surface.

9. The implant of claim 1, wherein the interstitial hydrogel portion has a crystallinity of 20% or greater.

10. (canceled)

11. A method of forming an implant having a cellulose-reinforced hydrogel, comprising:
attaching a cross-linked cellulose nanofiber network to a top bearing surface of the implant;
infiltrating a hydrogel material within interstitial regions of the cross-linked cellulose nanofiber network to form the cellulose-reinforced hydrogel; and
heating the cellulose-reinforced hydrogel so that a water content of the cellulose-reinforced hydrogel is 20% or less.

12. The method of claim 11, wherein heating comprises heating the cellulose-reinforced hydrogel so that the water content of the cellulose-reinforced hydrogel is 11% or less.

13. The method of claim 11, wherein heating comprises heating the cellulose-reinforced hydrogel so that the water content of the cellulose-reinforced hydrogel is 5% or less.

14. The method of claim 11, wherein the hydrogel material comprises polyvinyl alcohol (PVA).

15. The method of claim 11, wherein the cellulose-reinforced hydrogel is heated to a temperature ranging from 90-140° C.

16.-19. (canceled)

20. A method of implanting a resurfacing implant, the method comprising:
forming an opening in a bone;
inserting the resurfacing implant into the opening in the bone so that a bearing surface of the resurfacing implant faces away from the bone, wherein the bearing surface comprises a cellulose-reinforced hydrogel having a water content of 20% or less, wherein the cellulose-reinforced hydrogel comprises a cross-linked cellulose nanofiber network that is impregnated with a polyvinyl alcohol (PVA) hydrogel; and
allowing the cellulose-reinforced hydrogel to rehydrate in situ to have a water content of greater than 30%.

21. The method of claim 20, wherein allowing the cellulose-reinforced hydrogel to rehydrate comprises swelling the resurfacing implant to seal an edge of the resurfacing implant adjacent to the bone.

22. The method of claim 20, further comprising dehydrating the resurfacing implant prior to inserting.

23. The method of claim 20, wherein the cellulose-reinforced hydrogel has a crystallinity of 20% or more.
